(19) United States
(12) Patent Application Publication
(10) Pub. No.: US 2023/0148869 A1
(43) Pub. Date: May 18, 2023

(54) MIXED ULTRASOUND TRANSDUCER ARRAYS

(71) Applicant: DeepSight Technology, Inc., Los Altos, CA (US)

(72) Inventors: Danhua ZHAO, San Jose, CA (US); Jiangang ZHU, University City, MO (US); Lan YANG, Clayton, MO (US)

(21) Appl. No.: 17/990,596

(22) Filed: Nov. 18, 2022

Related U.S. Application Data
(63) Continuation of application No. PCT/US2021/033715, filed on May 21, 2021.
(60) Provisional application No. 63/029,044, filed on May 22, 2020.

(51) Int. Cl. A61B 5/00; B06B 1/06

(52) U.S. Cl. CPC A61B 5/0097; B06B 1/0607; A61B 2562/04

(57) ABSTRACT

An ultrasound device may include ultrasound transducer array that include one or more array elements of a first type and one or more array elements of a second type different from the first type. The first type may include a transducer configured to transmit acoustic waves. The second type may include an optical sensor. The array elements of the first and second types are configured to detect acoustic echoes corresponding to the transmitted acoustic waves.

### CROSS-REFERENCE TO RELATED APPLICATIONS

This application claims priority to U.S. Patent Application No. 63/029,044 filed on May 22, 2020, which is incorporated herein in its entirety by this reference.

### TECHNICAL FIELD

The present disclosure generally relates to the field of ultrasound, and in particular to methods and devices that enable ultrasound transducing using a mixed array including an array of optical sensors and other transducers.

### BACKGROUND

Ultrasound transducers are used in various industries including medical imaging and medical diagnosis due to a number of advantages. For example, ultrasound transducing utilizes ultrasound signal which has a remarkable penetration depth. Moreover, ultrasound imaging is known to be an advantageously non-invasive form of imaging, as it is based on non-ionizing radiation.

Various known ultrasound transducers used in ultrasound imaging have numerous drawbacks. For example, some ultrasound transducers are made of piezoelectric material, such as lead zirconate titanate (PZT). However, the 6 dB bandwidth of PZT materials is generally limited to only about 70%. Certain composite PZT materials have a slightly increased bandwidth, but still only achieve a bandwidth of up to about 80%. As another example, single crystal materials have increasingly been used in an effort to improve performance of ultrasound probes, but have lower Curie temperatures and are brittle. Another type of transducer material is silicon, which can be processed to build Capacitive Micromachined Ultrasound Transducer (CMUT) probes that can have increased bandwidth. However, CMUT probes are not very sensitive or reliable. Moreover, CMUT probes have several operational limitations. For example, CMUT probes are nonlinear transducers and, therefore, are not generally suitable for harmonic imaging. In addition, CMUT probes require an additional bias voltage to operate properly. Thus, there is a need for new and improved devices and methods for ultrasound transducing.

### SUMMARY

Generally, in some embodiments, an apparatus for imaging a target may include an ultrasound transducer array that includes one or more array elements of a first type and one or more array elements of a second type different from the first type. The first type may be a transducer (e.g., piezoelectric transducers or capacitive micromachined ultrasonic transducers (CMUT)) configured to transmit acoustic waves and the second type may be an optical sensor (e.g., an interference-based optical sensor such as an optical resonator, an optical interferometer, etc.). The array elements of the first and second types are configured to detect acoustic echoes corresponding to the transmitted acoustic waves.

In some variations, the ultrasound transducer array may include one or more rows in an elevation dimension. The ultrasound transducer array may, for example, include an odd number of rows or an even number of rows.

In some variations, the one or more array elements of the first type and the one or more array elements of the second type are in alternating rows. In some configurations, at least a portion of the array elements of the first type may be in a center row. In some configurations, at least a portion of the array elements of the second type may be in a center row.

The array elements may be arranged in an array with various suitable spacing from one on another. For example, in some variations, at least one row has a pitch that is larger than half of a wavelength of a center frequency of the transducer (e.g., in the row). In some variations, at least one row may have a pitch that is smaller than or equal to half of a wavelength of a center frequency of the transducer (e.g., in the row.) In some variations, array may include rows with equal pitch in the lateral dimension. Alternatively, in some variations, the ultrasound transducer array may include at least one row with a first pitch in the lateral dimension, and at least one row with a second pitch different than the first pitch in the lateral dimension. For example, in some variations, the ultrasound transducer array may include an inner row having the first pitch, and a row adjacent to the inner row having the second pitch, wherein the second pitch may be larger than the first pitch. In some variations, the inner row with the first pitch may include one or more array elements of the first type and the row adjacent to the inner row with the second pitch may include one or more array elements of the second type.

Furthermore, in some variations, pitch may vary within a row, as the ultrasound transducer array may include at least one row with variable pitch in the lateral dimension. For example, the at least one row with variable pitch may include a central region having a first pitch, and a lateral region adjacent to the central region having a second pitch larger than the first pitch. In some variations, the ultrasound transducer array may include a first row with a first variable pitch pattern in the lateral dimension, and a second row with a second variable pitch pattern in the lateral dimension, wherein the second variable pitch pattern may be different than the first variable pitch pattern. For example, in some variations, the ultrasound transducer array may include an inner row with the first variable pitch pattern comprising one or more array elements of the first type, and a row adjacent to the inner row with the second variable pitch pattern comprising one or more array elements of the second type.

The array elements of the first and second types may be arranged in different rows of the ultrasound transducer array. For example, in some variations, the ultrasound transducer array may include at least one row comprising at least one array element of the first type and at least one array element of the second type. The at least one row comprising at least one array element of the first type and at least one array element of the second type may be a center row. In some variations, the center row has a single array element of the second type. The single array element of the second type may include an optical sensor that can be about equal to or smaller than a wavelength of the transmitted acoustic waves.

In some variations, array elements of the first and second types may be arranged in the same row of the ultrasound transducer array. For example, in some variations, the ultrasound transducer array may include a center row including a set of array elements of the first type and a set of array elements of the second type. The array elements of the second type may, for example, be sized to be about equal to or smaller than a wavelength of the transmitted acoustic wave. In some variations, the ultrasound transducer array may include two or more rows, each of the two or more rows including at least one array element of the first type and at least one array element of the second type. The array elements of the second type may be spatially distributed in a regular pattern. The array elements of the second type may be spatially distributed in an irregular pattern. In some variations, the ultrasound transducer array may include at least 31 rows, at least some of the 31 rows comprising at least one array element of the first type and at least one array element of the second type. In some variations, the ultrasound transducer array may include a single row, the single row comprising at least one array element of the first type and at least one array element of the second type.

In some variations, the ultrasound transducer array may include a set of sub-apertures. The set of sub-apertures may include a first sub-aperture and a second sub-aperture, the first sub-aperture comprising a greater number of rows than the second sub-aperture. In some variations, the first sub-aperture may be a central sub-aperture and the second sub-aperture may be adjacent to the central sub-aperture. In some variations, at least one sub-aperture may include at least one array element of the first type and/or at least one array element of the second type.

In some variations, the ultrasound transducer array may include a first set of array elements of the first type and a second set of elements of the second type, wherein the first set of array elements and the second set of array elements are each in a sparse array configuration. The spatial distribution of the first set of array elements may be different from the spatial distribution of the second set of array elements.

The ultrasound transducer array may be on a substrate or other suitable surface. In some variations, the ultrasound transducer array may be on a planar surface. In some variations, the ultrasound transducer array may be on a curved surface. The curved surface may be a parabolic curve, a hyperbolic curve, or an elliptic curve.

In some variations, the ultrasound transducer array may include at least one annular array element. The ultrasound array may include a circular array element of the second type concentric with the at least one annular array element. In some variations, the at least one annular array element may be of the first type.

The ultrasound transducer array may be a 1 dimensional (1 D) array, a 1.25 dimensional (1.25 D) array, a 1.5 dimensional (1.5 D) array, a 1.75 dimensional (1.75 D) array, or a 2 dimensional (2D) array.

In some variations, the one or more array elements are optical sensors embedded in a polymer structure. The optical sensor may be optically coupled to an optical fiber to transmit a set of optical signals to a photodetector. The optical sensor may be configured to transmit the set of optical signals in response to the acoustic echoes.

### BRIEF DESCRIPTION OF THE DRAWINGS

FIG. 1 is a block diagram of an exemplary ultrasound imaging system with a mixed ultrasound transducer array.

FIG. 2 is a schematic illustration of an exemplary mixed ultrasound transducer array.

FIG. 3 is a schematic illustration of an exemplary mixed ultrasound transducer array.

FIG. 4 is a schematic illustration of an exemplary mixed ultrasound transducer array.

FIG. 5 is a schematic illustration of an exemplary mixed ultrasound transducer array.

FIG. 6 is a schematic illustration of an exemplary mixed ultrasound transducer array.

FIG. 7 is a schematic illustration of an exemplary mixed ultrasound transducer array.

FIG. 8 is a schematic illustration of an exemplary mixed ultrasound transducer array.

FIG. 9 is a schematic illustration of an exemplary mixed ultrasound transducer array.

FIG. 10 is a schematic illustration of an exemplary mixed ultrasound transducer array.

FIG. 11 is a schematic illustration of an exemplary 1 D mixed ultrasound transducer array.

FIG. 12 is a schematic illustration of an exemplary 2D mixed ultrasound transducer array.

FIG. 13 is a schematic illustration of an exemplary 2D mixed ultrasound transducer array.

FIG. 14 is a schematic illustration of an exemplary annular mixed ultrasound transducer array.

FIG. 15 is a schematic illustration of an exemplary mixed ultrasound transducer array.

FIG. 16 depicts elevation beam patterns of exemplary 1 D mixed ultrasound transducer arrays.

FIG. 17 depicts elevation beam patterns of exemplary 1 D and 1.5 D mixed ultrasound transducer arrays.

FIG. 18 is an illustrative schematic of an exemplary beam pattern.

### DETAILED DESCRIPTION

Non-limiting examples of various aspects and variations of the invention are described herein and illustrated in the accompanying drawings.

Described herein are ultrasound probes that have a mixed ultrasound transducer array including array elements of multiple different types. Mixed arrays described herein include one or more array elements of a first type and one or more array elements of a second type (e.g., optical sensors, interference-based optical sensors, optical resonators, optical interferometers, etc.) different from the first type. Optical sensors, such as, for example, WGM optical resonators, may have high sensitivity and broad bandwidth in reception of ultrasound signals compared to other types of ultrasound sensors. The one or more array elements of the first type (e.g., transducers) may be used to form a first image. In parallel, the one or more array elements of the second type (e.g., the optical sensors) are used to detect acoustic echoes that can be used to form a second image. The second image that is generated by highly sensitive and broadband optical sensors may be used independently or can be combined with the first image to form an even further improved image. Because of the high sensitivity and broad bandwidth of optical sensors, the image produced by the optical sensors may have improved spatial resolution, improved penetration depth, improved signal-to-noise ratio (SNR), improved tissue harmonic imaging, and/or improved Doppler sensitivity.

Optical sensors described herein may include an interference-based optical sensor(s), such as an optical resonator(s), an optical interferometer(s), etc. The optical resonators may include, for example, a whispering galley mode (WGM) optical resonator(s), a microbubble optical resonator(s), a microsphere resonator(s), a micro-toroid resonator(s), a micro-ring resonator(s), a micro-disk optical resonator(s), and/or the like.

The optical resonators may include a closed loop of a transparent medium that allows some permitted frequencies of light to continuously propagate inside the closed loop, and to store optical energy of the permitted frequencies of light in the closed loop. For example, the optical resonators may permit propagation of whispering gallery modes (WGMs) traveling the concave surface of the optical resonators and corresponding to the permitted frequencies to circulate the circumference of the resonator. Each mode from the WGMs corresponds to propagation of a frequency of light from the permitted frequencies of light. The permitted frequencies of light and the quality factor of the optical resonators described herein may be based at least in part on geometrical parameters of the optical resonator, refractive index of the transparent medium, and refractive indices of an environment surrounding the optical resonator.

The optical interferometers may include a Mach-Zehnder interferometer(s), a Michelson interferometer(s), a Fabry-Perot interferometer(s), a Sagnac interferometer(s), and/or the like. For example, a Mach-Zehnder interferometer may include two nearly identical optical paths (e.g., fibers, on-chip silicon waveguides, etc.). The two optical paths may be finely adjusted acoustic waves (e.g., by physical movement caused by the acoustic waves, tuning of refractive index caused by the acoustic waves, etc.) to effect distribution of optical powers in an output(s) of the Mach-Zehnder interferometer, and therefore, detect a presence or a magnitude of the acoustic waves.

As further described herein, the optical sensors may be coupled to the outside world to receive light, to transmit light, and to be useful in practice (e.g., for an ultrasound imaging or other transducing application in an acousto-optic system). Acousto-optic systems based on optical sensors may directly measure ultrasonic waves through the photo-elastic effect and/or physical deformation of the resonator(s) in response to the ultrasonic waves (e.g., ultrasonic echoes). For example, in the presence of ultrasonic (or any pressure) waves, the WGMs traveling an optical resonator may undergo a spectral shift caused by changes in the refractive index and shape of the optical resonator. The spectral change can be easily monitored and analyzed in spectral domain and light transmission intensity to and from the optical resonator. Additional spatial and other information can furthermore be derived by monitoring and analyzing shifting WGMs among multiple optical resonators. Exemplary mixed ultrasound arrays are described herein.

### Dimensionality of Mixed Arrays

The mixed ultrasound transducer array, such as those described herein, may have various dimensionalities. For example, the mixed array can be configured for operation in a 1 dimensional (1 D) configuration, a 1.25 dimensional (1.25 D) array configuration, a 1.5 dimensional (1.5 D) array configuration, a 1.75 dimensional (1.75 D) array configuration, or a 2 dimensional (2D) array configuration, as described in further detail below. Generally, dimensionality of the ultrasound transducer array relates to the range of elevation beam width (or elevation beam slice thickness) that is achievable when imaging with the ultrasound transducer array, and how much control the system over the transducer array's elevation beam aperture size, foci, and/or steering throughout an imaging field (e.g., throughout imaging depth). A 1 D array has only one row of elements in elevation dimension and a fixed elevation aperture size. A 1.25 D array has multiple rows of elements in elevation dimension and a variable elevation aperture size, but a fixed elevation focal point via an acoustic lens. A 1.5 D array has multiple rows of elements in elevation dimension, a variable elevation aperture size, and a variable elevation focus via electronical delay control. A 1.75 D array is a 1.5 D array with additional elevation beam steering capability. A 2D array has large numbers of elements in both lateral and elevation dimensions to satisfy the minimum pitch requirement for large beam steering angles.

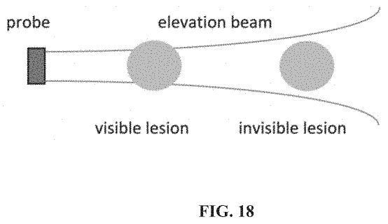

FIG. 18 shows a schematic of an exemplary beam pattern from an ultrasound probe. Having an appropriate beam width can be important in medical imaging. For example, if a lesion like cancer tissue is larger than the elevation beam width, it may be detected (“visible lesion”). Otherwise, if the lesion is smaller than the elevation beam width, the lesion may not be detected (“invisible legion”). This is because in general, lesions have hypo-echogenicity relative to their surrounding tissues, and therefore are shown as darker regions in the image. If the elevation beam is wider than a lesion, the echo signals generated from surrounding tissues of the lesion within the beam width can fill in the dark lesion and make it invisible or hard to see. Accordingly, the ability to control one or more parameters (e.g., elevation aperture size, elevation foci, etc.) of the elevation beam can provide greater control of ultrasound imaging and/or lead to better image quality in certain circumstances.

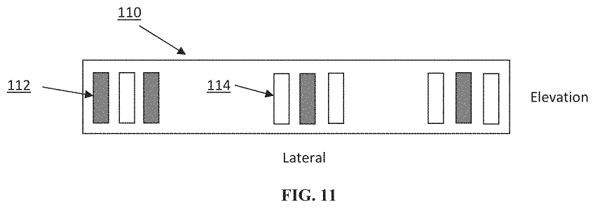

A 1 D array has only one row of elements in the elevation dimension, and a fixed elevation aperture size. In other words, a 1 D array has multiple array elements arranged in only one row extending in one dimension (i.e., the lateral dimension). For example, as shown in FIG. 11, array elements in a 1 D array may be arranged in a single row that is linear along the lateral dimension only, but does not extend in the elevation dimension. In some variations of a linear array, the spacing between two adjacent elements may be equal to about one wavelength of the transmitted acoustic wave. In some variations of a phased array, the spacing between two adjacent elements may be about half the wavelength of the transmitted acoustic wave. The single row of array elements in the transducer array means no range in the elevation dimension. Accordingly, a 1 D array has both a fixed elevation aperture size and a fixed elevation focus, and a thin slice thickness in the elevation dimension cannot be maintained throughout the imaging depth. In addition to this slice thickness limitation, a 1 D array has an elevation aperture that is a compromise between near field performance and far field performance.

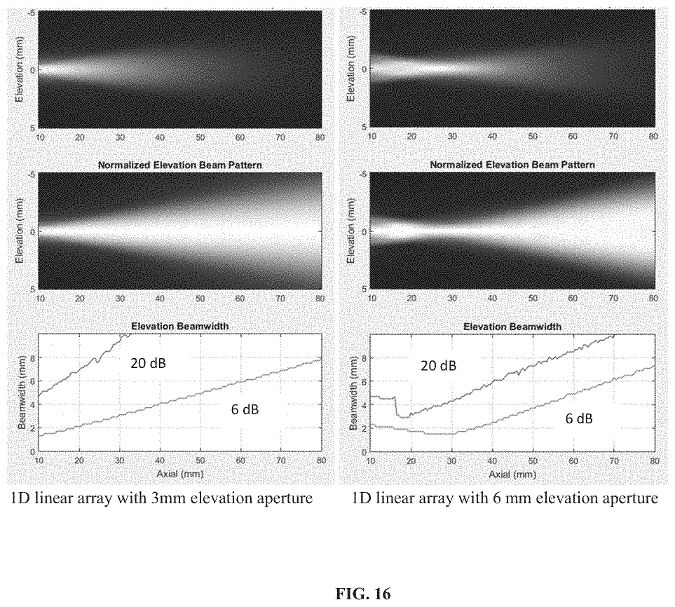

FIG. 16 provides a comparison between a linear 1 D array with 3 mm elevation aperture and a linear 1 D linear array with 6 mm elevation aperture. Specifically, FIG. 16 depicts beam patterns (top row) and normalized beam patterns (middle row) for the two 1 D arrays for varying image depth. FIG. 16 also depicts plots (bottom row) that represent 6 dB and 20 dB elevation beam width versus image depth ranging from about 10 mm to about 80 mm. As shown in FIG. 16, the elevation beam width of the 3 mm aperture array generally linearly increases with image depth, for both the 6 dB and 20 dB elevation beams. However, the elevation beam width of the 6 mm aperture array decreases from 10 mm to about 22 mm, remains flat until 31 mm before starting to increase linearly with depth. Thus, a 1 D configuration may have some limitations in imaging, but may nevertheless be useful in certain applications (e.g., certain imaging depths).

A 1.25 D array has multiple rows of elements in the elevation dimension and a variable elevation aperture size, but a fixed elevation focal point via an acoustic lens. The variable elevation aperture size can be electronically controlled, for example. Varying the elevation aperture size enables some control over the narrowing of the elevation beam width, meaning the ultrasound system can achieve more appropriate overall elevation beam slice thickness. The elevation beam width can further be reduced by adding more rows in the array in the elevation dimension. However, while a 1.25 D array has variable elevation aperture size, it has fixed elevation foci, such that beam thickness cannot be controlled throughout the imaging field (e.g., imaging depth).

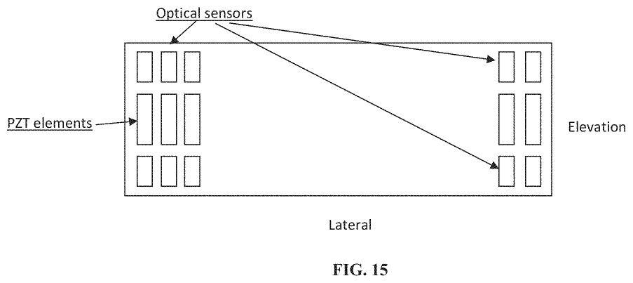

A 1.5 D array has multiple rows of elements in the elevation dimension, a variable elevation aperture size, and a variable elevation focus via electronical delay control. The variable elevation aperture size and one or more variable elevation foci can, for example, be electronically controlled. For example, FIG. 15 illustrates an exemplary 1.5 D mixed array having at least two elements (e.g., two rows) in the elevation dimension. In some variations, the mixed array may include an odd number of rows to allow for symmetry across a single center row in the mixed array. In some variations, the mixed array may include an even number of rows. The mixed array has one or more array elements of a first type (e.g., PZT transducer) and one or more array elements of a second type (e.g., optical resonators, optical interferometers, etc.) as described above. The 1.5 D array shown in FIG. 15 includes three rows for sake of illustration, but it should be understood that the array may include any suitable number of rows. These three rows are arranged adjacent to one another in the elevation dimension: one inner (center) row having one or more array elements of the first type and two outer rows having one or more array elements of the second type. The spacing between these elements in the elevation dimension may be larger than one wavelength of the transmitted acoustic wave. In some variations, the 1.5 D array includes linear arrays and each of the inner row and the two outer rows can have enough elements to satisfy a minimum pitch requirement of one wavelength of the transmitted acoustic wave. Alternatively, in some variations, the 1.5 D array may be a phased array and each of the inner row and the two outer rows may have a pitch of half a wavelength of the transmitted acoustic waves. The inner row and the two outer rows can have the same pitch or different pitch. For example, the inner row may include 128 transducer elements while the two outer rows may include 32 transducer elements.

As described above, both the elevation aperture size and elevation foci of the 1.5 D array can be controlled. In some variations, the number of array elements may be larger than the number of channels in the imaging system, and so in these variations the system may include one or more analog switches (e.g., high voltage switches) to select desired sub-apertures of a 1.5 D array. Since both its elevation aperture size and elevation foci can be selectively adjusted, the 1.5 D array may be controlled to selectively achieve a narrower elevation beam width throughout the imaging field, and enable the ultrasound probe to image smaller lesions in addition to larger legions at various imaging depths.

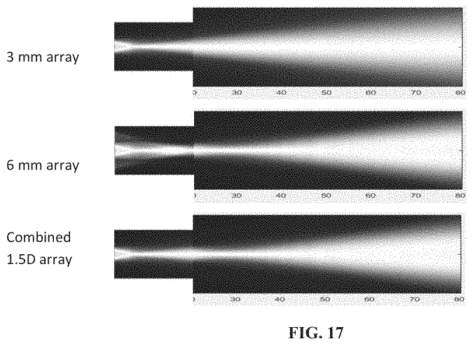

FIG. 17 illustrates a comparison of elevation beam patterns of exemplary 1 D and 1.5 D mixed arrays. The top two images of FIG. 17 show beam patterns for the linear 1 D array with 3 mm elevation aperture and the linear 1 D linear array with 6 mm elevation aperture discussed above with respect to FIG. 16. The top two images of FIG. 17 are similar to those shown in FIG. 16, except that FIG. 17 also shows very near field beam patterns (from 0 to 10 mm) to illustrate a more complete beam pattern profile from 0 to 80 mm of image depth. The linear 1 D array with 3 mm elevation aperture (top row) has thinner elevation beam width in the near-field from 0 mm to about 20 mm, but thicker elevation beam width in the far-field after about 20 mm. The linear 1 D array with 6 mm elevation aperture (middle row) has thinner elevation beam width between about 20 mm to about 40 mm, but thicker elevation beam width above about 20 mm. However, a 1.5 D array with variable elevation aperture size and elevation foci (bottom row) has a thinner elevation beam width across a greater image depth range than either of the individual 1 D arrays; that is, across at least the very near field from about 0 mm to about 40 mm, for example. In other words, the 1.5 D array can achieve better overall elevation beam width throughout the imaging field. The elevation beam width can be further narrowed by adding additional elements to the array in the elevation dimension.

Accordingly, the 1.5 D array can have number of advantages compared to a 1 D array. First, the 1.5 D array can have thinner elevation beam slice thickness that can help to resolve small structures such as, for example, tiny blood vessels and small cysts. Second, the 1.5 D array can have better and more uniform image quality for images spanning from a near-field image to a far-field image. Lastly, the 1.5 D array can have a better detail resolution that the 1 D array without sacrificing penetration and sensitivity.

A 1.75 D array is a 1.5 D array, but with additional elevation beam steering capability. In other words, a 1.75 D array is similar to the 1.5 D array, in that the 1.75 D array includes multiple rows of elements in the elevation dimension, a variable elevation aperture size, and a variable elevation focus. However, the 1.75 D array may be electronically controllable to enable some degree of freedom in beam steering (e.g., up to about 5 degrees in at least one direction, up to about 10 degrees in at least one direction, up to about 15 degrees in at least one direction, or up to about 20 degrees in at least one direction). Like the 1.5 D array, a system incorporating a 1.75 D array may include one or more analog switches to select desired sub-apertures of the array.

Finally, a 2D array has large numbers of elements in both lateral and elevation dimensions to satisfy the minimum pitch requirement for large beam steering angles. For example, a 2D array includes multiple array elements arranged in both the lateral and elevation dimensions, and is electronically controllable to enable a full suite of variable elevation aperture, variable elevation foci, and full beam steering control. Like the 1.5 D array, a system incorporating a 2D array may include one or more analog switches to select desired sub-apertures of the array.

Ultrasound Imaging System with Mixed Ultrasound Transducer Arrays

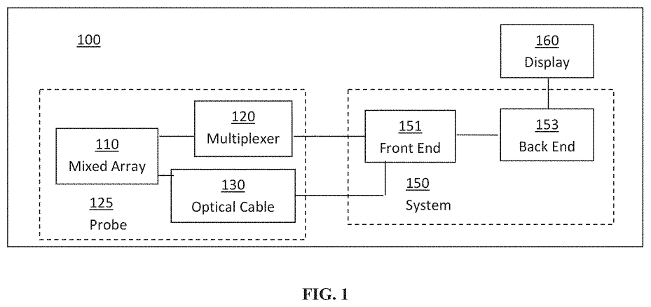

FIG. 1 is a block diagram of an exemplary ultrasound imaging system 100 with a mixed array (also referred to herein as “mixed ultrasound transducer array”). The ultrasound imaging system includes a probe 125, an imaging system 150, and a display 160. The probe 125 can be connected (without intervening components) or coupled (with or without intervening components) to the imaging system 150. The probe 125 can receive and/or transmit a set of signals (e.g., electrical signals, acoustic signals, optical signals, etc.) from/to the imaging system 150. The imaging system 150 can be connected (without intervening components) or coupled (with or without intervening components) to the display 160. The imaging system 150 can receive and/or transmit a set of signals (e.g., electrical signals, electromagnetic signals, etc.) from/to the display 160.

The probe 125 includes a mixed array 110, a multiplexer 120, and an optical cable 130. The mixed array 110 includes one or more array elements of a first type capable of transmitting acoustic waves (e.g., piezoelectric transducers) and one or more array elements of a second type that are highly sensitive with broadband response (e.g., WGM optical resonators). The mixed array 110 includes an array of transducer elements and may be configured for operation in a 1 dimensional (1 D) configuration, a 1.25 dimensional (1.25 D) array configuration, a 1.5 dimensional (1.5 D) array configuration, a 1.75 dimensional (1.75 D) array configuration, or a 2 dimensional (2D) array configuration, as further described below. The one or more array elements of the first type in the mixed array 110 can be operatively coupled to the multiplexer 120. The one or more array elements of the second type in the mixed array 110 can be operatively coupled to the optical cable 130.

In some variations, the probe 125 can be configured to iteratively scan across a field of view by using a phased array of the mixed array 110. Doing so will generate line-by-line images using the one or more array elements of the first type and/or the one or more array elements of the second type. Synthetic Aperture (SA) algorithms can then be used to generate a high-resolution image. Additionally or alternatively, in some variations, the probe 125 can be configured to use different patterns of acoustic excitation such as, for example, using a first group of transducer elements to transmit acoustic waves, while using a second group of transducer elements or all transducer elements to receive ultrasound echoes that correspond to the acoustic waves.

The mixed array 110 may include a large number (e.g., 10, 100, 200, 1000, 2000, 10,000, and/or the like) of elements. In some variations, the array may be arranged in a rectangular configuration and may include N×M elements, where N is the number of rows and M is the number of columns. The mixed array may include one or more array elements of a first type and one or more array elements of a second type, where the first type may be a transducer configured to transmit ultrasound waves and the second type may be an optical sensor (e.g., an optical resonator, an optical interferometer, etc.). The one or more array elements of the first type and the one or more array elements of the second type may be collectively positioned in a rectangular arrangement, a curved arrangement, a circular arrangement, or a sparse array arrangement. Various example configurations of array elements in the mixed array 110 are described in further detail below.

The transducer(s) in the mixed array 110 may include, for example, a lead zirconate titanate (PZT) transducer(s), a polymer thick film (PTF) transducer(s), a polyvinylidene fluoride (PVDF) transducer(s), a capacitive micromachined ultrasound transducer(s) (CMUT), a piezoelectric micromachined ultrasound transducer(s) (PMUT), a photoacoustic sensor(s), a transducer(s) based on single crystal materials (e.g., LiNbO₃(LN), Pb(Mg_{1/3}Nb_{2/3})—PbTiO₃ (PMN—PT), and Pb(IninNb_{1/2})—Pb(Mg_{1/3}Nb_{2/3})—PbTiO₃ (PIN—PMN—PT)), and/or any transducer suitable for acoustic transducing.

The optical sensor may be, for example, a microbubble resonator, a fiber-based resonator, an integrated photonic resonator, a micro-disk resonator, a Fabry-Perot interferometer, and/or the like. For example, in some implementations, the optical sensor may include an optical microbubble resonator. The optical microbubble resonator can made of an optically transparent material such as, for example, glass, transparent polymer, silicon nitride, titanium dioxide, or any other material that is suitably optically transparent at an operation wavelength of the optical microbubble resonator. The optical microbubble resonator includes an outer microbubble surface with a radius (R) and an inner microbubble surface with a radius (r), thereby defining a resonator wall thickness equivalent to (R-r). A set of resonant frequencies (due to propagation of a set of WGMs) of the optical microbubble resonator can have high quality factors suitable for highly sensitive transducing probes. In general, a sensitivity of optical resonators can be improved by increasing the quality factor of the optical resonator. In particular, in such implementations, the sensitivity can be controlled by a wall thickness (R— r) of the optical microbubble resonator. When used as ultrasound detectors, the optical microbubble resonator can have a low noise equivalent pressure and a broadband operation bandwidth as described in further detail herein. In some implementations, optical sensors may include sensing nodes formed at a cross-section of optical fibers and optical waveguides when light propagating in the optical waveguides couples in the optical fibers and propagates in circumferences of the optical fibers. In some variations the optical sensor may include an integrated photonic optical resonator. For example, in some variations the optical sensor may be similar to any of the optical resonators described in U.S. Patent App. Nos. 62/945,538 and 63/001,738, each of which is incorporated herein in its entirety.

The space inside and/or around the optical sensors may be filled with an ultrasonic enhancement material, such as for example, polyvinylidene fluoride, parylene, polystyrene, and/or the like. The ultrasonic enhancement material can increase sensitivity of the optical sensors. For example, the ultrasonic enhancement material can have a relatively high elasto-optic coefficient, such that in response to the optical sensors receiving a set of ultrasound echoes, the refractive index of the ultrasonic enhancement material changes more than the refractive index of the material of a material(s) of the optical sensors (e.g., upon receiving a mechanical stress or strain induced by the set of ultrasound echoes).camera

The optical cable 130 may include a dedicated optical path for transmitting and/or receiving optical signals to/from the optical sensors. The optical cable 130 may include a fiber optical cable(s) or a coax cable(s). A choice of the optical cable 130 may depend upon a type of the optical signals. An array of the optical sensors of the mixed array 110 can be linearly arranged on a substrate. The array of the optical sensors may be equidistant from each other. Additionally or alternatively, at least some optical sensors in the array can be separated by different distances. In some configurations, the array of the optical sensors can all be optically coupled to a single optical waveguide. Accordingly, signals from the multiple the optical sensors can be coupled to and communicated by a single optical waveguide. In some configurations, the array of the optical sensors can be optically coupled to an array of optical waveguides. Accordingly, optical signals from the array of the optical sensors can be coupled to and communicated by multiple optical waveguides in the optical cable 130 to the imaging system 150.

The multiplexer 120 may include analog switches. The analog switches may include a large number of high voltage analog switches. Each analog switch can be connected to an individual system channel. As a result, the multiplexer 120 may selectively connect an individual system channel from a set of system channels of the imaging system 150 to a transducer of the mixed array 110. Accordingly, electrical signals from the one or more array elements of a first type can be coupled to and communicated by multiple optical waveguides in the optical cable 130 to the imaging system 150.

The imaging system 150 may include a frontend system 151 and a backend system 153. Generally, the frontend system 151 may include at least two components including a transmit beamformer and receive beamformer. The transmit beamformer and the receive beamformer may include multiple transmit channels and receive channels that are connected (e.g., via a set of electrical wires, via a set of optical waveguides, and/or the like) to the one or more array elements of the first type and/or the one or more array elements of the second type. For example, the transmit beamformer may include 128 transmit channels connected to the multiplexer 120 and the receive beamformer may include 256 receive channels connected to the optical cable 130 and/or the multiplexer 120. The frontend system may further include a set of photodetectors to convert optical signals to electrical signals. The backend system 153 may include a processor to process signals received from the mixed array 110 to generate images, a memory operatively coupled to the processor to store the images, and a communication interface to present the images to a user (e.g., via graphical user interface).

The display 160, may be operatively coupled to the backend system 153 of the imaging system 150 to display a set of images generated by the imaging system 150. In some variations, the display 160 may include an interactive user interface (e.g., a touch screen) and be configured to transmit a set of commands (e.g., pause, resume, and/or the like) to the imaging system 150. In some variations, the ultrasound imaging system 100 may further include a set of ancillary devices (not shown) used to input information to the ultrasound imaging system 100 or output information from ultrasound imaging system 100. The set of ancillary device may include, for example, a keyboard(s), a mouse(s), a monitor(s), a webcam(s), a microphone(s), a touch screen(s), a printer(s), a scanner(s), a virtual reality (VR) head-mounted display, a joystick(s), a biometric reader(s), and/or the like (not shown).

### Example Configurations of Mixed Arrays

Described below are various exemplary configurations of array elements in a mixed ultrasound transducer array. As described above, the mixed ultrasound transducer array may generally include one or more array elements of a first type and one or more array elements of a second type, where one or more array elements of the first type may include a set of transducers (e.g., piezoelectric transducer, single crystal material transducer, piezoelectric micromachined ultrasound transducer (PMUT), or capacitive micromachined ultrasonic transducer (CMUT), etc.) and the one or more array elements of the second type may include a set of optical sensors.

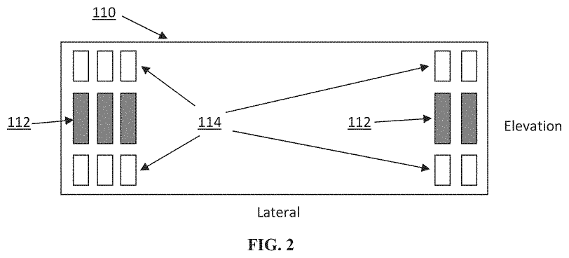

In some variations, the ultrasound transducer array may include one or more rows in an elevation dimension. For example, the array elements may be collectively positioned in a rectangular array including a number of rows and a number of columns. In some variations, as shown in FIG. 2, the mixed array may include 3 rows of elements in an elevation dimension. The 3 rows include an inner row and two outer rows. The two outer rows may be made of the second type 114 (e.g., optical sensors). The second type 114 may include, for example, a set of microbubble resonators, a set of fiber-based resonators, a set of integrated photonic resonators, a set of micro-disk resonators, a set of optical interferometers, and/or the like. The inner row may be made of the first type 112 (also referred to herein as “transducers”). The first type 112 may include, for example, a lead zirconate titanate (PZT) transducer(s), a polymer thick film (PTF) transducer(s), a capacitive micromachined ultrasound transducer(s) (CMUT), and/or any transducer suitable for acoustic transducing.

The two outer rows may include equal number of elements that are positioned in parallel in a corresponding column. Each pair of elements 114 positioned in the same column in the two outer rows may be optionally connected (e.g., electrically connected or electromagnetically coupled) to form a single combined outer element for a 1.25 dimensional (1.25 D) array configuration or a 1.5 dimensional (1.5 D) array configuration.

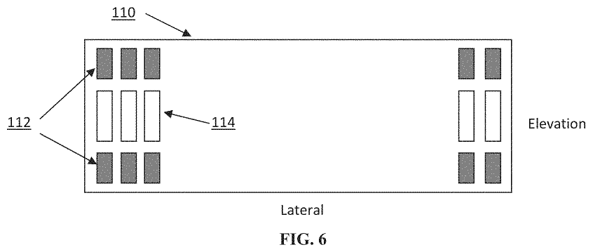

Although FIG. 2 depicts a transducer array having three rows, in some variations, the number of rows may be any odd number such as 3, 5 . . . 2n+1, where n is an integer. In some variations, array elements of the first type 112 may be arranged in the center row of a set of an odd number of rows. For example, a 1.5 D array configuration may include 5 rows with a PZT transducer row in the center row, two optical sensor rows adjacent to the center row, and two PZT transducer rows on the outermost rows adjacent to the optical sensor rows. Having the center row include transducers may be advantageous in some variations. For example, since the center row includes transducer elements of first type 112 that can perform both transmission and reception of ultrasound waves, the elevation beam profile does not have a “dip” in the middle, for both the transducers' transmission mode and reception mode. This dip occurring in an elevation beam profile can degrade image quality and introduce image artifacts. Accordingly, arranging transducer elements of the first type 112 in the center row (e.g., as shown in FIG. 2) may advantageously help avoid such degradation in image quality and image artifacts. However, in some variations, such as that shown in FIG. 6, the mixed transducer array may include optical sensors in a center row.

In some variations, the one or more array elements of the first type 112 (e.g., transducers) and the one or more array elements of the second type 114 (e.g., optical sensors) may be in alternating rows. For example, FIG. 2 shows an exemplary variation in which array elements of the first type 112 alternates with the second type of array elements 114, with the first type 112 in a center row. As another example, FIG. 6 shows an exemplary variation in which the first type of array elements 112 alternates with the second type of transducers, with second type of array elements 114 in a center row.

In some variations, the spacing between adjacent array elements (pitch) may be selected for certain performance parameters. Pitch may be defined as a distance between the center of a transducer element and the center of an adjacent transducer element. In some variations, the pitch may measure larger than half a wavelength of an operational frequency of the acoustic waves (e.g., transmitted by the piezoelectric transducers), such as when the array is in a phase array operation. In some variations, the pitch may measure larger than a full wavelength of the operational frequency of the acoustic waves, such as when the array is in a linear array operation. In some variations, the pitch may measure smaller than a half wavelength of the operational frequency of the acoustic waves or may measure smaller than a full wavelength of the operational frequency of the acoustic waves.

In some variations, all rows in a mixed array may have the same pitch in the lateral dimension (e.g., as shown in FIG. 2). However, in some variations, at least some of the rows in a mixed array may have different pitch in the lateral dimension. In other words, one row may have a first pitch, and another row may have a second pitch, wherein the second pitch is either smaller or larger than the first pitch.

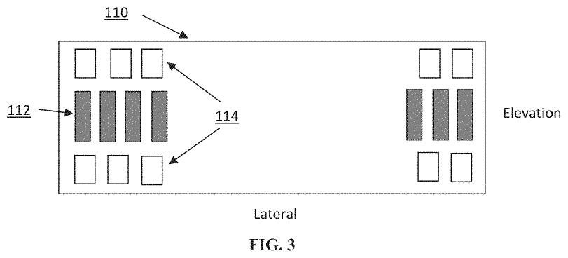

For example, as shown in FIG. 3, a mixed array 110 may include an inner (center) row having a first uniform pitch, and two outer rows having a second uniform pitch different from the first uniform pitch. In some variations, the second uniform pitch may be larger than the first uniform pitch (i.e., spaced farther apart from one another). The two outer rows may include optical sensors 114. The inner row may include a first type 112 that are transducers such as, for example, a PZT transducer(s), a CMUT transducer(s), and/or the like. In some variations, one of the two outer rows may have the second uniform pitch and the other of the two outer rows have a third uniform pitch different from both the first and second pitches. In some variations, the mixed array may include a set of rows with any odd number of rows, such as 3, 5 . . . 2n+1 rows, where n is an integer. In such variations, each row from the set of rows may have a unique pitch that is different from pitch of any other rows. In some implementations, the set of rows may have an ascending pitch starting from a center row having the smallest pitch among the set of rows and steadily increasing to the outer rows having the largest pitch among the set of rows.

In ultrasound imaging, a transducer pitch is generally selected based on the operating frequency. More specifically, the transducer pitch may be inversely proportional to an operating frequency (e.g., proportional to an operating wavelength corresponding to the operating frequency) to avoid grating lobes. When imaging superficial tissues, small elevation apertures and high frequencies are often used. On the other hand, large elevation apertures and low frequencies may be advantageous for imaging of deep tissues.

Due to the varying pitch among its rows, the mixed array shown and described with respect to FIG. 3 may advantageously perform both imaging of superficial tissues and imaging of deep tissues. In this variation, the center row with smaller pitch may be used to produce high resolution of the superficial tissues using a high operating frequency. For imaging the deep tissues, all rows (including rows with larger pitch) may be used to produce high penetration images using a low operating frequency.

Another advantage of using larger pitch is the option of reducing the overall number of transducer elements in the imaging system 150. A larger pitch results in fewer transducer elements per area and smaller areal density of cables (e.g., optical cables 130, coaxial cables, etc.) of the probe 125 that connect the mixed array 110 of the probe 125 to the frontend 151 of the imaging system 150. Therefore, a reduction in the number of transducer elements as shown and disclosed in this embodiment involves several advantages including: a reduced number of cables (i.e., a thinner cable bundle), lighter weight of the probe 125, and lower cost of manufacturing and operation.

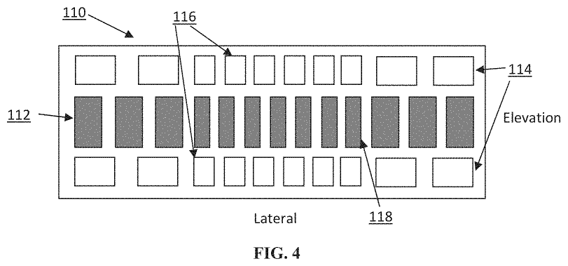

Additionally or alternatively, in some variations, the distance between transducer array elements (pitch) in a particular row may be the same or may vary along the length of the row. Using such variable pitch may be beneficial because it may generally enable imaging both superficial tissues and deep tissues, similar to that described above with respect to FIG. 3. FIG. 4 is a schematic description of an exemplary mixed array with at least one row with variable pitch in the lateral dimension. For example, in some variations, a row with variable pitch may include a central region that has a first pitch, and one or more lateral regions adjacent to the central region that has a second pitch different (e.g., smaller or larger) than the first pitch. Although only three groups (left, middle, and right) of pitch for each row are illustrated in FIG. 4, in some variations, two, or four or more groups of pitch can be used as well. In some variations, as shown in FIG. 4, the center row and the two outer rows may each have variable pitch (instead of the uniform pitch as disclosed with respect to FIG. 2 or FIG. 3). For example, the ultrasound transducer array may include a first row with a first variable pitch pattern in the lateral dimension, and a second row with a second variable pitch pattern in the lateral dimension. The second variable pitch pattern may be different than the first variable pitch pattern. Additionally or alternatively, two or more rows in the mixed array may each include the same or similar variable pitch pattern along their lengths in the lateral dimension.

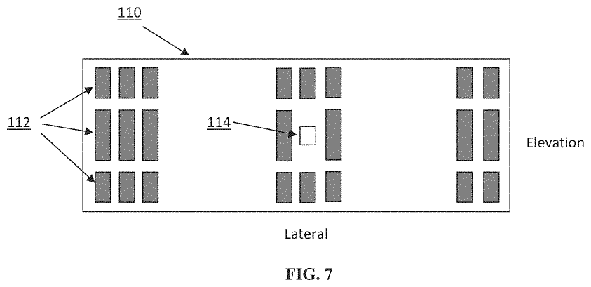

In some variations, the one or more array elements of the first type and the one or more array elements of the second type may be collectively arranged in a set of sub-groups, or sub-apertures of the mixed array 110. For example, in some variations, the mixed array 110 may be divided into a set of sub-apertures each having a set of one or more rows. Each sub-aperture may include a set of transducer elements from the one or more array elements of the first type and/or one or more array elements of the second type. In some variations, the mixed array may be configured in a 1.5 dimensional (1.5 D) array that includes three rows (e.g., three rows of elements as shown in FIG. 7). In some variations, only a central transducer element of the set of transducer elements in a sub-aperture positioned at the center of the mixed array is an optical sensor 114. In such variations, the rest of the transducer elements are made of different types of transducers 112 that may include, for example, a lead zirconate titanate (PZT) transducer(s), a polymer thick film (PTF) transducer(s), a CMUT transducer(s), and/or any transducers suitable for acoustic transducing. In some implementations, a size of the optical sensor is small (e.g. comparable to or smaller than an operating wavelength of acoustic waves and/or acoustic echoes).

Each sub-aperture may include a different number of rows than one or more other rows in the set of rows. Each row may include a uniform pitch or variable pitch. For example, the mixed array 110 may include five sub-apertures having one row, three rows, five rows, three rows, and one row, consecutively from a left-most aperture to a right-most aperture of the five sub-apertures.

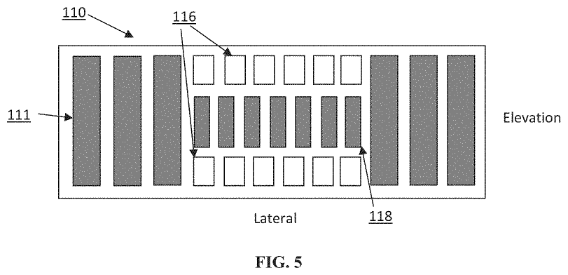

For example, FIG. 5 is a schematic description of an exemplary mixed array 110 with sub-apertures. The mixed array 110 shown in FIG. 5 may be divided into three sub-apertures including: a left sub-aperture, a middle sub-aperture, and a right sub-aperture. Both of the left sub-aperture and the right sub-apertures 110 may have only one row that is depicted as having a uniform pitch, but it should be understood that in some variations these sub-apertures may include a row that is variable pitch. The middle sub-aperture, however, may have multiple rows, such as an inner row 118 and two outer rows 116. The inner row 118 may have a certain pitch and the two outer rows 116 may have a different pitch from the inner row. Any of these pitches in the middle sub-aperture may be uniform or variable. The two outer rows 116 may include optical sensors. All the other rows, including the inner row of the middle sub-aperture, the left sub-aperture, and the right sub-aperture, may include other transducers such as, for example, a PZT transducer(s), a CMUT transducer(s), etc.

FIG. 6 is a schematic description of an exemplary mixed array that is similar to the mixed array of FIG. 2, except only relative positions of the array elements of the first type 112 and the array elements second type 114 are switched. In a similar way, relative positions of the array elements of the first type 112 and the array elements of the second type 114 may be switched in the mixed array of FIG. 3, the mixed array of FIG. 4, and the mixed array of FIG. 5, and/or any other mixed array shown and described herein.

FIG. 7 is a schematic description of an exemplary mixed array. The mixed array may include one or more array elements of a first type including a set of transducers and one or more array elements of a second type including a set of optical sensors. The mixed array may include at least one row that has at least one array element of the first type and at least one array element of the second type. As shown in FIG. 7 for example, the mixed array may, for example, include a center row including at least one array element of the first type and at least one array element of the second type. For example, the center row may have a single array element of the second type, while the other rows may only have array elements of the first type. The single array element of the second type can be an optical sensor that is about equal to or smaller than a wavelength of the transmitted acoustic waves. In some variations, the use of an optical sensor can minimize the complexity of probe manufacturing while utilizing an ultra-high sensitivity of the optical sensor (e.g., an ultra-high sensitivity of a WGM optical resonator) for image quality improvement.

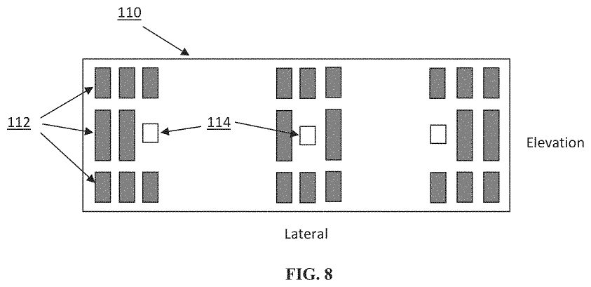

FIG. 8 is a schematic description of an exemplary mixed array 110. A mixed array may include two or more rows including a center row. The center row may include multiple elements of the first type 112 and multiple array elements of the second type 114. The array elements of the second type may be about equal to or smaller than a wavelength of the transmitted acoustic wave. At least one transducer element in the inner row is an optical sensor 114 and the rest of the elements are array elements of the first type 112 that may include, for example, a PZT transducer(s) and/or a CMUT transducer(s). Similar to the mixed array 110 of FIG. 7, in some implementation, a size of the optical sensors in the mixed array 110 of FIG. 8 can be comparable to or smaller than an operating wavelength of acoustic waves and/or acoustic echoes. In some implantations, a size of the optical sensors may be larger than the operating wavelength of the acoustic waves and/or the acoustic echoes.

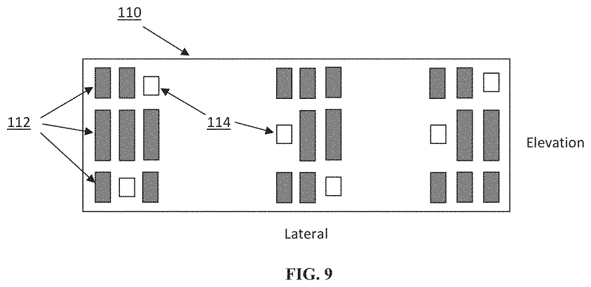

FIG. 9 is a schematic description of an exemplary mixed array. An ultrasound transducer array may include two or more rows. Each of the two or more rows may have at least one array element of the first type and at least one array element of the second type. The array elements of the second type may be spatially distributed in a regular pattern or may be spatially distributed in an irregular pattern (e.g., random pattern). A set of the elements on the inner row and two outer rows may include optical sensors 114 and the rest of the elements include the first type 112 that including, for example, a PZT transducer(s) and/or a CMUT transducer(s). In some configurations, a spatial distribution of positions of the optical sensors 114 can be random. In some configurations, a spatial distribution of positions of the optical sensors 114 can follow a dispositioning pattern (e.g., be the same, shift to the right by one cell among sensor elements, shift to down by two cells among sensor elements). A size of an optical sensor can be smaller than or the same as the size of the first type 112.

Variations of mixed arrays as described in FIGS. 7-9 in which may involve multiple advantages. For example, each row in these mixed arrays having both array elements of both types can, to some extent, both transmit acoustic waves (e.g. ultrasound waves) and detect acoustic echoes (e.g., ultrasound echoes), thereby enabling a more distributed imaging functionality. Additionally, the distributed optical sensors can increase an overall and more distributed sensitivity of the mixed array to acoustic echoes. Furthermore, the mixed array of these variations may generate two independent images that are generated by the one or more array elements of the first type and the one or more array elements of the second type, and an imaging system (such as the imaging system 150 as shown and described with respect to FIG. 1) can then combine the two independent images to form a single combined image that is improved compared to each of the two independent images.

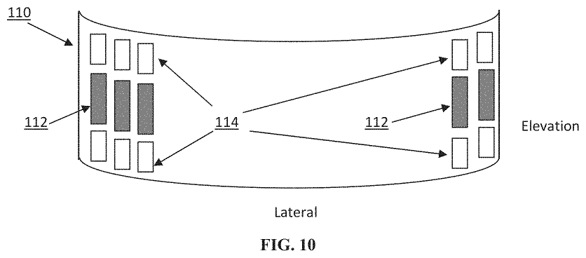

Although the mixed arrays shown in FIGS. 2-9 are generally shown as on a planar substrate or surface, it should be understood that a mixed array may be arranged on any other suitable surface (e.g., curved surface). For example, FIG. 10 is a schematic description of an exemplary mixed array 110 on a curved substrate. The mixed array 110 may include one or more array elements of a first type including a set of transducers and one or more array elements of a second type including a set of optical sensors on a curved surface, such as on a curved panel. The curved surface may include a profile of a circular curve, a quadratic curve (e.g., parabolic curve, a hyperbolic curve, an elliptic curve, etc.) and/or any curve suitable for ultrasound imaging. A curvature of the panel can be in the direction of the field of view of the mixed array 110. In some configurations, the curvature is a concave curvature. In some configurations, the curvature is a convex curvature. The mixed array may include three rows of elements in elevation dimension as shown in FIG. 10. The three rows may include an inner row and two outer rows. In some variations, the two outer rows may include array elements of the second type 114 (e.g., optical sensors) and the inner row may include array elements of the first type 112 (e.g., PZT transducers, CMUT transducer, and/or the like). In some variations, the mixed array may include any odd number of rows such as 3, 5 . . . 2n+1, where n is an integer. In such variations, an inner row of the mixed array may include array elements of the first type 112 and the rest of rows thereafter may alternate between array elements of the second type 114 and the first type 112.

The positioning of transducer elements in the mixed array of FIG. 10 is similar to the positioning of transducer elements in the mixed array of FIG. 2 except that the mixed array of FIG. 10 is mounted on a curved panel. Positioning of the mixed array of FIG. 10 can be achieved by multiplying a transformation matrix corresponding to the curvature of the mixed array of FIG. 10 to a matrix of positions of the mixed array of FIG. 2. The relative positions of transducer elements in the mixed arrays of any of FIGS. 2-13 can be transformed to be mounted on a curved panel.

FIG. 11 is a schematic description of an exemplary 1 D mixed array 110 that includes a single row including multiple array elements or transducer elements. The multiple array elements may include at least one array element of the first type 112 (e.g., PZT transducers, CMUT transducer, and/or the like) and at least one array element of the second type 114 (i.e., optical sensors). In some configurations, the spatial distribution of those of the first type 112 and those of the second type 114 may be random. In some configurations, the spatial distribution of the array elements of the first type 112 and the array elements of the second type 114 may follow a dispositioning pattern. Compared to a traditional 1 D array that includes only one type of transducer, the mixed array (including a set of optical sensors, as shown in FIG. 11) may have an improved performance in sensing bandwidth and/or sensitivity due to the addition of optical sensors such as, for example, optical resonators (e.g., a WGM optical resonator, a microbubble optical resonator, a microsphere resonator, a micro-toroid resonator, micro-ring resonators, or a micro-disk optical resonator, and/or the like).

Although FIGS. 2-11 depict transducer arrays having odd number such as 1, 3, 5 . . . 2n+1 number of rows, where n is an integer, in some variations, the transducer arrays may have even number of rows such as 2, 4, 6, . . . 2n number of rows. For example, in some variations, the mixed array 110 may have two rows of the array elements of the first type 112 (CMUT transducers, PMUT transducers, and/or the like) and two rows of elements of the second type 114 (e.g., optical resonators, optical interferometers, and/or the like).

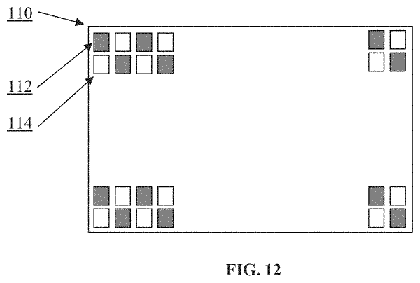

FIG. 12 is a schematic description of an exemplary 2D mixed array 110 arranged in a rectangular configuration and may include N×M transducer elements, where N is the number of rows and M is the number of columns and are both integers. In some implementations, the number of rows and/or the number of columns may be greater than 31 rows and/or 31 columns. For example, a 2D mixed array may include 64×96=6,144 transducer elements. The mixed array may include one or more array elements of a first type and one or more array elements of a second type. The one or more array elements of the first type may include a set of transducers and the one or more array elements of the second type may include a set of optical sensors. The one or more array elements of the first type and the one or more array elements of the second type may be collectively positioned in a rectangular arrangement. In some configurations, the spatial distribution of the first type 112 and the second type 114 may be random. In some configurations, the spatial distribution of the first type 112 and the second type 114 may follow a dispositioning pattern. Compared to a traditional 2D array that includes only one type of transducer, the mixed array (including a set of optical sensors, as shown in FIG. 12) may demonstrate an improved performance in sensing bandwidth and/or sensitivity due to the addition of optical sensors.

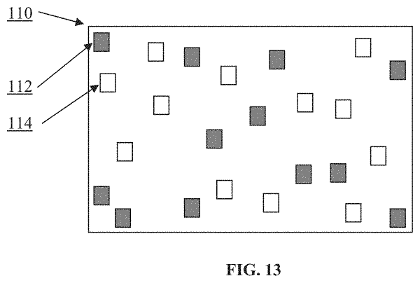

FIG. 13 is a schematic description of an exemplary 2D mixed array 110 in a sparse array configuration. Arranging the mixed array 110 in the sparse array configuration instead of a fully sampled arrangement (such as the arrangement as shown and described with respect to FIG. 12) may reduce total number of transducer elements used to manufacture the mixed array. For example, a sparse 2D array having the same size of a fully sampled 2D (as shown and described with respect to FIG. 12), may include only 1000 transducer elements compared to the 64×96=6,144 transducer elements of in the fully sampled mixed array of FIG. 12. The mixed array may include one or more array elements of a first type and one or more array elements of a second type. The one or more array elements of the first type may include a set of transducers and the one or more array elements of the second type may include a set of optical sensors. The one or more array elements of the first type and the one or more array elements of the second type may be collectively positioned in a sparse array configuration. In some configurations, the spatial distribution of array elements of the first type 112 and the array elements of the second type 114 may be random. In some configurations, the spatial distribution of the array elements of the first type 112 and the second type 114 may follow a statistical distribution (e.g., a normal distribution, a Gaussian distribution, and/or the like). By using the sparse spatial distribution of array elements of the first type 112 and the second type 114, generation of grating lobes in an image produced by the mixed array may be reduced/prevented. A spatial distribution of the array elements of the first type 112 may be the same, similar, or different from, a spatial distribution of the array elements of the second type 114. For example, a first set of positions of a set of optical sensors in the mixed array 110 may have a random spatial distribution and second set of positions of a set of PZT transducers in the mixed array 110 may have a normal distribution.

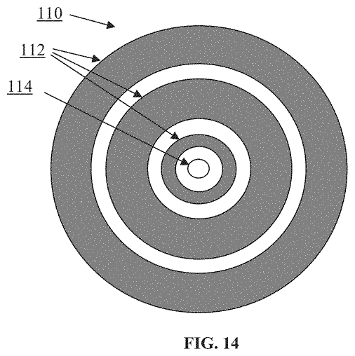

Although the mixed arrays described above with respect to FIGS. 2-13 are primarily described in terms of a rectangular arrangement with one or more rows, it should be understood that other array shapes may similarly be mixed with multiple types of array elements 112 and 114. For example, FIG. 14 is a schematic description of an exemplary annular mixed array 110. An annular array can generally produce improved acoustic (e.g., ultrasound) beam patterns in three dimensional space because of its symmetric shape. Improved acoustic (e.g., ultrasound) beam patterns may result in better image quality of an acoustic imaging systems (e.g., medical ultrasound imaging systems) since the image quality is highly correlated with acoustic beam patterns.

Like the arrays described above, the annular mixed array may include one or more array elements of a first type 112 and one or more array elements of a second type 114. The one or more array elements of the first type may include a set of transducers and the one or more array elements of the second type may include a set of optical sensors. The mixed array may include at least one circular array element and at least one annular array element arranged around and concentric with the circular array element. For example, as shown in FIG. 14, the mixed array may include at least one circular optical sensor 114 (e.g., a ring resonator) in the center of the mixed array and a set of annular transducers 112 (such as, for example, a PZT transducer(s) and/or a CMUT transducer(s)) arranged around and concentric with the circular optical sensor in order of increasing diameter. While the variation shown in FIG. 14 includes three annular array elements around a circular array element, it should be understood that the mixed array can include any suitable number of annular array elements, such as two, three, four, five, or more than five annular or ring-shaped elements. Furthermore, in some variations, the one or more array elements of the first type and the one or more array elements of the second type may both be annular elements collectively arranged in a concentric configuration.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. An apparatus for imaging a target, comprising:
an ultrasound transducer array comprising:
one or more array elements of a first type, wherein the first type is a transducer configured to transmit acoustic waves; and
one or more array elements of a second type different from the first type, wherein the second type is an optical sensor,
wherein the array elements of the first and second types are configured to detect acoustic echoes corresponding to the acoustic waves.

2-17. (canceled)

18. The apparatus of claim 1, wherein the ultrasound transducer array comprises at least one row comprising at least one array element of the first type and at least one array element of the second type.

19-23. (canceled)

24. The apparatus of claim 18, wherein the ultrasound transducer array comprises two or more rows, each of the two or more rows comprising at least one array element of the first type and at least one array element of the second type.

25-28. (canceled)

29. The apparatus of claim 1, wherein the ultrasound transducer array comprises a plurality of sub-apertures.

30-31. (canceled)

32. The apparatus of claim 29, wherein each sub-aperture comprises at least one array element of the first type.

33. The apparatus of claim 32, wherein each sub-aperture further comprises at least one array element of the second type.

34-52. (canceled)

53. The apparatus of claim 1, wherein the optical sensor is an interference-based optical sensor.

54. The apparatus of claim 53, wherein the optical sensor comprises an optical resonator or an optical interferometer.

55. (canceled)

56. A method of ultrasound transducing comprising:
transmitting acoustic waves using an ultrasound probe, the ultrasound probe comprising an ultrasound transducer array having one or more array elements of a first type and one or more array elements of a second type different from the first type; and
receiving acoustic echoes in response to the acoustic waves, using the one or more array elements of the first type and the one or more array elements of the second type,
wherein the one or more array elements of the second type are optical sensors.

57-72. (canceled)

73. The method of claim 56, wherein the ultrasound transducer array comprises at least one row comprising at least one array element of the first type and at least one array element of the second type.

74-83. (canceled)

84. The method of claim 56, wherein the ultrasound transducer array comprises a plurality of sub-apertures.

85. The method of claim 84, wherein each sub-aperture comprises at least one array element of the first type.

86. The method of claim 85, wherein each sub-aperture further comprises at least one array element of the second type.

87-107. (canceled)

108. The method of claim 56, wherein the optical sensors are interference-based optical sensors.

109. The method of claim 108, wherein the optical sensors comprise at least one of an optical resonator and an optical interferometer.
